(19) United States
(12) Patent Application Publication
(10) Pub. No.: US 2017/0151086 A1
(43) Pub. Date: Jun. 1, 2017

(54) CUSTOM MADE ORAL APPLIANCE FOR AIRWAY MANAGEMENT OF THOSE WITH OBSTRUCTIVE SLEEP APNEA (OSA)

(71) Applicant: Behzad M. Fareid, Kingsburg, CA (US)

(72) Inventor: Behzad M. Fareid, Kingsburg, CA (US)

(21) Appl. No.: 14/956,309

(22) Filed: Dec. 1, 2015

(51) Int. Cl. A61F 5/56

(52) U.S. Cl. CPC A61F 5/566

(57) ABSTRACT

An oral appliance for managing a person's airway and for a more efficient breathing is disclosed. The oral appliance provides greater comfort by maintaining an open air passage through the mouth and by preventing the tongue from blocking the windpipe during sleep. The appliance has a mouthpiece that includes partial tooth trays capable of adjustable multidirectional movements relative to each other. The disclosed oral appliance maximizes the space available for forward and side to side movements of the tongue.

### CROSS-REFERENCE(S) TO RELATED APPLICATION(S)

None.

### TECHNICAL FIELD

This application relates generally to an oral appliance. More specifically, this application relates to a therapeutic device placed in a person's mouth for sleep airway management and prevention of sleep apnea.

### BRIEF DESCRIPTION OF THE DRAWINGS

The drawings, when considered in connection with the following description, are presented for the purpose of facilitating an understanding of the subject matter sought to be protected.

FIG. 1 shows a perspective view of a typical prior art oral appliance for sleep apnea.

FIG. 2 shows a perspective view of an embodiment of the disclosed oral appliance.

FIG. 3 shows a perspective view of another embodiment of the disclosed oral appliance.

FIG. 4 shows a plan view of an embodiment of the disclosed oral appliance.

FIG. 5 shows a cross-sectional view of the upper tooth partial trays of an embodiment of the disclosed oral appliance.

FIG. 6 shows a cross-sectional view of the lower tooth partial trays of another embodiment of the disclosed oral appliance.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description often references specific materials such as stainless steel, acrylics, plastics, custom or non-custom made or pre-fabricated connectors such as wires, it will be appreciated that the disclosure may include other types of soft or hard and flexible, semi-flexible, or inflexible materials or made by CAD-CAM design and manufacturing.

Sleep apnea is not merely a condition of snoring or loud breathing during sleep, and thus is more than an annoyance to those sleeping nearby. The term apnea refers to the momentary (10 seconds or more in duration is considered an apnea) cessation of breathing and sleep apnea is a momentary cessation of breathing during sleep. Severe sleep apnea can deprive the sleeper of sufficient oxygen for a restful sleep, and the sudden cessation will usually cause the sleeper to awake. Several episodes a night will deprive the sleeper of sufficient sleep to function alertly the next day. Yet, such sleep apnea is generally a chronic condition, and it can be difficult to find a solution in many cases.

Sleep apnea may have various causes, such as the momentary blockage of the windpipe by the tongue, which may occur particularly when the sleeper is already breathing through his her mouth. While this may or may not be fatal, it nevertheless creates an apneic episode and interrupts sound sleep. As a result, a number of devices have been developed in the past to treat such episodes of sleep apnea, ranging from mechanical devices intended for placement in the mouth to restrain movement of the tongue to more complex and costly devices, such as Continuous Positive Airway Pressure machines (CPAP, APAP or BPAP) and other electronic devices. The more costly and complex electronic devices are beyond the means of many people who can benefit from such devices. Research has showed that patients have more tolerance and use for oral appliance than for CPAP. Furthermore, Oral appliance yields better results than surgical intervention.

Obstructive sleep apnea occurs when the muscles (of the airway) in the back of the throat relax. These muscles support the soft palate, the triangular piece of tissue hanging from the soft palate (uvula), the tonsils, the side walls of the throat and the tongue. When the muscles relax, the airway narrows or closes as one breathes in, and cannot get an adequate breath. This will eventually lower the level of oxygen in one's blood.

Brain senses this inability to breathe and briefly wakes the person up so that she can reopen her airway. This awakening is usually so brief that the person, in most cases, does not remember it. A person with sleep apnea may make a snorting, choking or gasping sound. This pattern can repeat itself five to 30 times or more each hour, all night long. The highest apneic episodes occurs during supine positions and in REM stage. These disruptions impair one's ability to reach the desired deep, restful phases of sleep, and she will probably feel sleepy during the waking hours. People with obstructive sleep apnea may not be aware that their sleep was interrupted. In fact, some people with this type of sleep apnea think they sleep well all night.

Oral appliances that are used to alleviate snoring and obstructive sleep apnea are devices worn in the mouth, similar to orthodontic retainers or sports mouthguards. They have several advantages over other forms of therapy. Oral appliances are relatively inexpensive, easy to travel with, non-invasive, easy to fabricate, and quite well accepted by patients. The appliance works non-invasively to assist in keeping the airway open and its effectiveness has been superior to when it is compared with various invasive surgical interventions for the treatment of Obstructive Sleep Apnea (OSA).

Oral appliance therapy is becoming recognized by the dental and medical community as an effective treatment option in the management of (obstructive) sleep breathing disorders. Medical and dental researchers have joined together to evaluate and improve the outcome of the use of oral appliances. It is generally recommended that oral appliances be used in patients with primary snoring or mild (to moderate) obstructive sleep apnea and inpatients who are intolerant of or refuse treatment with continuous positive airway pressure devices (CPAP).

Oral appliances fall into two basic categories: tongue-retaining devices (TRD) and mandibular advancing devices (MAD). Tongue-retaining devices are small acrylic (silicone) devices which fit by suction on the tip (anterior portion) of the tongue by various controlled and measurable mechanical means. Mandibular advancing devices are small acrylic devices that fit over the upper and lower teeth and advance the lower jaw as well as increase the vertical opening of patient's mouth in order to facilitate airway patency during various stages of sleep. In addition, it facilitates advancing of the mandible. Both move the base of the tongue forward and open the airway. In general, TRDs do not work as well as MADs. They are non-invasive, easy to use and are effective in improving breathing, reducing snoring and reducing sleep apnea. They are less effective for treating people with moderate or severe obstructive sleep apnea and people who are obese. MAD may be used in combination with CPAP due to better compliance and in order to keep the airway patent when patient fails to use CPAP; particularly, with patients that fail to utilized their CPAP on regular basis as prescribed for them.

TRD's are typically custom-made appliances with an anterior bulb that holds the tongue in a forward position during sleep by means of negative pressure (or suction). Currently, the TRD appears to be the appliance of choice for patients who have few or no teeth and for patients who have large tongues. In addition, the TRD is a good appliance for patients who cannot adequately advance their lower jaw for whatever reason. Its disadvantages are that it is more difficult for both clinicians and patients to use on a regular basis, it poses problems for patients who cannot breathe through their nose, it irritates the end of the tongue over time and it is a single jaw and tongue position appliance.

MAD devices fit over both the upper and the lower teeth and move the lower jaw forward, which brings the tongue and some throat tissues along with it in order to prevent collapsing the airway. They also stimulate the pharyngeal muscles to maintain an open airway during REM sleep—the muscles that normally relax during various stages of sleep, including REM. There are several different designs for MADs. Some designs are not adjustable after fabrication and have to be remade if any alterations are required. If the devise if not adjustable, it cannot pass the FDA requirement for treatment of OSA and therefore cannot be used. Others have an excellent range of adjustment. MAD appliances, in general, require a healthy jaw joint (TMJ) and several healthy teeth with healthy gums in both the upper and lower jaw.

There is a need in the art for an oral appliance which mitigates the disadvantages of the prior art.

Briefly described, oral appliances are disclosed for keeping the tongue from obstructing the airway during sleep. In contrast to the prior art, these oral appliances provide a larger opening in frontal (anterior) portion of the mouth and a larger space inside the mouth for the tongue to move around. In addition, the disclosed designs allow adjustable forward movement of the lower jaw and maintains a distance between the upper and the lower teeth, as well as side by side relative movement of the upper and the lower jaws. In comparison with the prior art, these new apparatus create more space for the tongue to move around in all directions.

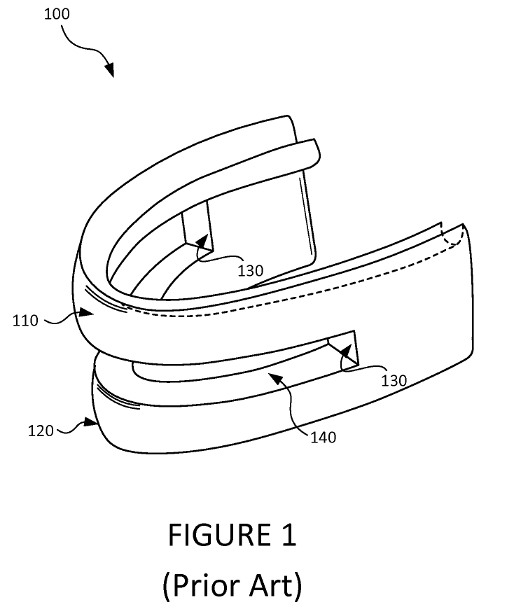

FIG. 1 shows a perspective view of a typical prior art oral appliance 100 for sleep apnea. The upper tray 110 and the lower tray 120 of such customary oral appliances are attached and fixed with respect to each other by a fixed connector wall 130 which also provides a gap 140 between the two trays for passage of air. The upper tray 110 and the lower tray 120 can have no relative movement with respect to each other. This shortcoming of the prior art oral appliances make them very restrictive and uncomfortable and does not allow for the natural movements of the upper and the lower jaws. Additionally, all typical oral appliances, as illustrated in FIG. 1, have full upper and lower trays that encase the entire sets of the upper and the lower teeth, and the front portions of the upper and the lower trays 110 and 120 interfere with the forward movements of the tongue and unnecessarily prevent adequate opening of the airway.

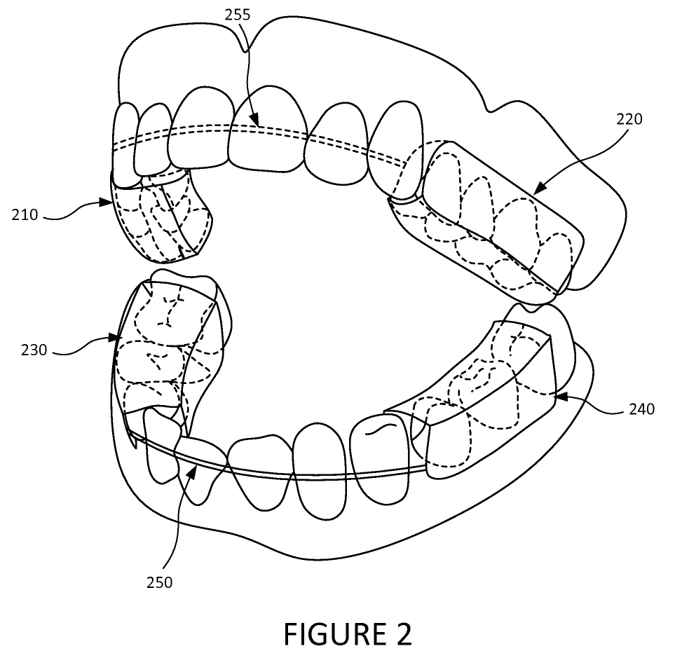

FIG. 2 shows a perspective view of an embodiment of the disclosed oral appliance. In this embodiment the upper tooth tray comprises two independent partial trays 210 and 220 and the lower tooth tray comprises two independent partial trays 230 and 240, which accommodate the molars and/or premolars. In some embodiments the two upper partial trays 210 and 220 and/or the two lower partial trays 230 and 240 are attached to each other by metal wires, bars or connectors 250 and 255. The length of connectors 250 and 255 may be adjustable or non-adjustable and they can either be placed on labial or the lingual or both areas. In some embodiments the metal wires, pre-fabricated or custom made bar or connectors, may be replaced by metal strips or other narrow connectors made from metal or non-metal materials. Because most embodiments of the presently disclosed oral appliance only have partial tooth trays and the upper and the lower partial trays are configured to have relative movements with respect to each other, the tongue will (have more freedom and open space in order to) be able to move forward and sideway almost as comfortably as if the user did not have any oral appliance in his/her mouth. In this embodiment each partial tray includes three planes that form an open channel and at least partially cover three sides of a tooth.

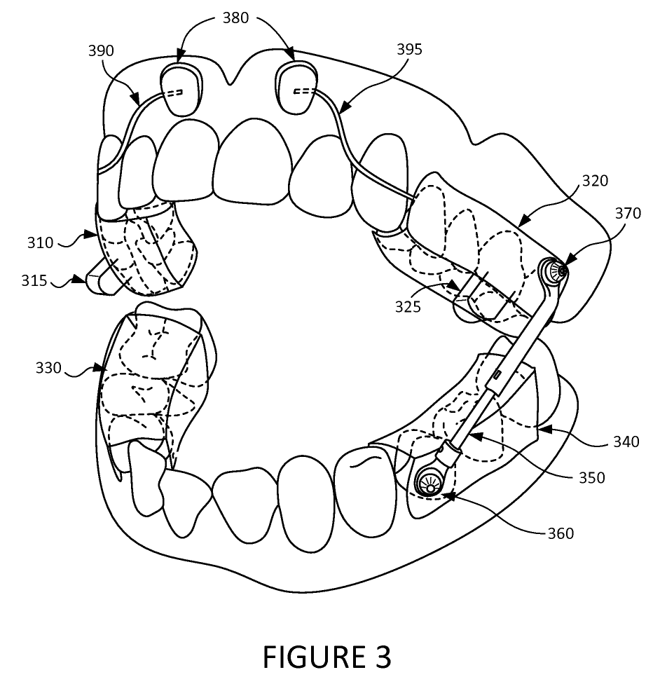

FIG. 3 shows a perspective view of another embodiment of the disclosed oral appliance. In this embodiment the upper partial side trays 310 and 320 have at least an adjustable or non-adjustable height protrusion 315 and 325, respectively, to prevent the upper and the lower trays from ever lying on each other and preventing the air from easily passing through. Another purpose of 315 and 325 is to produce more space for the tongue to spread around especially longitudinally and laterally to create a larger airway area for the patient to breath. Adjusting the height of the protrusions 315 and 325 will also adjust the vertical distance between the upper and the lower jaw as needed for each specific patient. For example protrusions 315 and 325 may be simply cut to a desired length for a desired vertical distance between the upper and the lower jaws. During any relative movement of the jaws, at least one of the protrusions 315 and 325 will stay on the lower partial tray 330 or 340 at all times, which keeps the upper and the lower jaws at a desired distance from each other at all times. This creates more space in the mouth for the tongue during sleep (in consideration for patient's TMJ physiological health).

In various embodiments the protrusion 315 and 325 are located such that to provide as much space for the side movements of the tongue as possible. In some embodiments the protrusions are fabricated on the lower partial trays and in other embodiments the protrusions are fabricated on both the lower and the upper partial trays. The protrusions may be of any desired dimensions, shapes and/or forms or even may be made from a material different from the material of the partial trays. In various embodiments only one protrusion may be fabricated on one of the upper or the lower partial trays, whereas in other embodiments multiple protrusions may be fabricated over the partial trays.

FIG. 3 also illustrates a small adjustable-length shaft or connector (Herbst Connector) 350 (only shown on one side of the mouth for clarity) which is flexibly connected to the rear side of the upper partial tray 320 and the front side of the lower partial tray 340 at hinge points 370 and 360, respectively. The “front,” “rear,” and “under” are all defined relative to a user's mouth. The hinge points 370 and 360 may each include a ball and socket joint. Adjustments of the length of shaft 350 on both sides of the mouth can move and keep the lower jaw forward or backward relative to the upper jaw, which provides an additional degree of freedom for managing the airway of the user that tend to grind their teeth in lateral directions. In the configuration of FIG. 3, lengthening of shaft 350 pushes the lower jaw forward and shortening of shaft 350 pulls the lower jaw backward with respect to the upper jaw by, for example, turning a part of shaft 350 around its longitudinal axis.

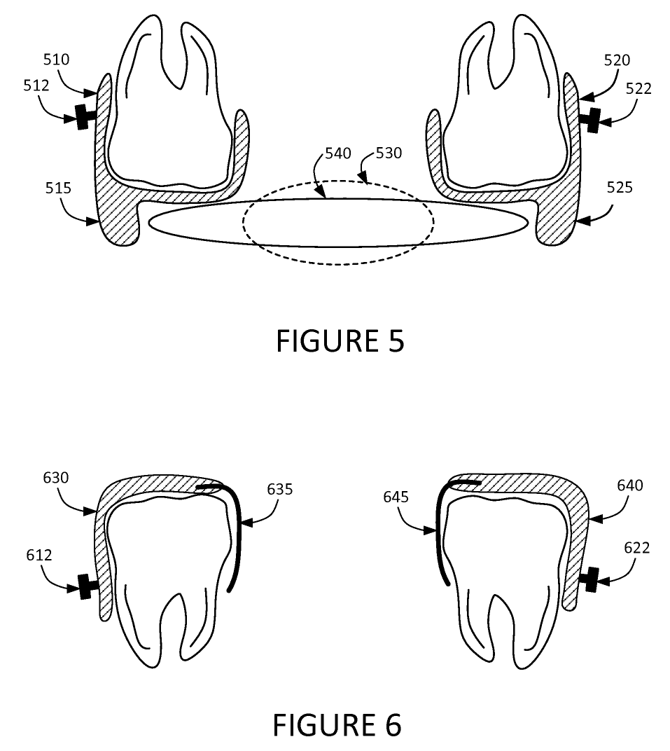

In some embodiments the shaft or connector 350 may be flexibly connected to the rear side of the lower partial tray 340 and the front side of the upper partial tray 320. In such embodiments lengthening of shaft 350 pushes the lower jaw backward and shortening of shaft 350 pulls the lower jaw forward with respect to the upper jaw. Shaft/connector 350 may or may not be used in some embodiments of the disclosed oral appliance. In various embodiments the Shaft/connector 350 may be replaced by springs, elastic bands, or other elastic, spring, or spring-loaded mechanisms and the hinge points may be replaced by connector hooks 512, 522, 612, and 622, shown in FIGS. 5 and 6. Such optional arrangements provides the user with more flexibility of back and forth movements of the lower jaw with respect to the upper jaw and with more control over the vertical distance of the jaws. Those skilled in the art are familiar with adjustable-length shafts/connectors and their various mechanisms. Pharyngometer, Rhinometer, “Snoring Sound Technique” and phonetic bites are some of the devices or techniques used in order to determine the final vertical and horizontal poisoning of the upper and lower trays.

In some embodiments, small plastic or acrylic pads 380 which are attached to the upper partial trays 310 and 320 by wires 390 and 395, respectively, may be placed between the upper lip of the user and his/her gum. This addition will further facilitate breathing from the nose.

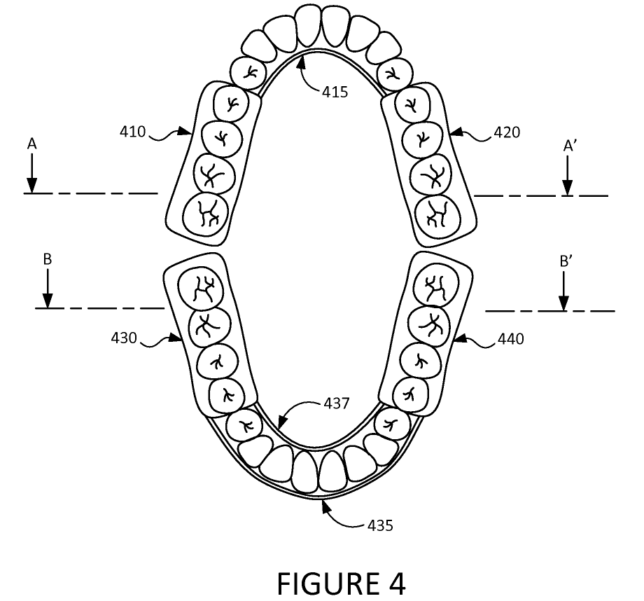

FIG. 4 shows a plan view of an embodiment of the disclosed oral appliance. As clearly illustrated in FIG. 4, the two upper partial trays 410, 420 and the two lower partial trays 430, 440 are attached to each other by the relatively stiff but narrow adjustable-length or nonadjustable-length wires 415, 435 and 437. FIG. 4 illustrates that the two upper partial trays 410, 420 or the two lower partial trays 430, 440 may be connected by one or two connectors and connectors may be placed in the labial, the lingual, or both areas. The negligible space taken by the narrow wire connection or pre-fabricated or custom made bar or connector 415 of the upper partial trays 410 and 420 and the negligible space taken by the narrow wire connections or pre-fabricated or custom made bars or connectors 435 and 437 of the lower partial trays 430 and 440 allow ample space for the tongue inside the user's mouth. In contrast, the prior art oral appliances and their trays are usually completely made of plastic or acrylic which takes more space for the same structural strength.

FIG. 5 shows a cross-sectional view A-A′ of an embodiment of the disclosed oral appliance such as the one illustrated in FIG. 4. As can be seen in FIG. 5, the plastic or acrylic partial trays 510 and 520 enclose three sides of the teeth and therefore grab the teeth from two sides. Protrusions 515 and 525, which in this embodiment are made from the same material as the rest of the trays, will cause the upper teeth to stay at a distance from the lower teeth and provide more space for the tongue 540 in comparison with a situation such as the one illustrated in FIG. 1 which causes a narrower space for tongue 530.

FIG. 6 shows a cross-sectional view B-B′ of an embodiment of the disclosed oral appliance such as the one illustrated in FIG. 4. As can be seen in FIG. 6, the plastic or acrylic parts of the partial trays 630 and 640 enclose only two sides of the teeth and the third side is held by retentive wires 635 and 645, respectively. As seen in FIG. 6, retentive wires 635 and 645 are partially embedded in the plastic or acrylic part of the tooth trays. Use of retentive wires as an extension of the partial trays inside the mouth will provide even more space for the tongue in comparison with the space available when the entire partial tooth tray is made of plastic or acrylic.

As clearly illustrated in FIG. 5, the cross-sectional profile of the partial tray is substantially a “U” shape and as clearly shown in FIG. 6, the cross-sectional profile of the partial tray may be substantially an “L” shape or a “U” shape if the cross-section includes retentive wire 635 or 645.

In various embodiments, springs, elastic bands or other elastics, springs, or spring-loaded mechanisms may either replace the Shaft/connector 350 or be installed in addition to the Shaft/connector 350. In such embodiments connector hooks 512, 522, 612, and 622, shown in FIGS. 5 and 6, may be used for the installation of the elastic, spring, or spring-loaded mechanism. Such optional arrangements provides the user with more flexibility for back and forth movements of the lower jaw with respect to the upper jaw and/or for preventing the appliance from opening more than it is required and thereby restricting the vertical opening of the appliance.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless the above detailed description section explicitly defines such terms. Accordingly, the actual scope of the claimed invention encompasses not only the disclosed embodiments, but also all equivalent ways of practicing or implementing the claimed invention.

The above specification, examples, and data provide a complete description of the manufacture and use of the composition of the invention. Since many embodiments of the invention can be made without departing from the spirit and scope of the invention, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. An oral appliance for facilitating breathing of a user, the oral appliance comprising:
a first and a second partial upper tooth trays for being placed over upper molars and/or premolars of the user;
a first and a second partial lower tooth trays for being placed over lower molars and/or premolars of the user, wherein the first partial lower tooth tray is substantially located under the first partial upper tooth tray and the second partial lower tooth tray is substantially located under the second partial upper tooth tray;
a first adjustable-length connector, and/or an elastic mechanism, attached between a rear side of the first partial upper tooth tray to a front side of the first partial lower tooth tray or between a front side of the first partial upper tooth tray to a rear side of the first partial lower tooth tray;
a second adjustable-length connector, and/or an elastic mechanism attached between a rear side of the second partial upper tooth tray to a front side of the second partial lower tooth tray or between a front side of the second partial upper tooth tray to a rear side of the second partial lower tooth tray; and
an adjustable or a non-adjustable height protrusion from each of the first and the second partial upper tooth trays or from the partial lower tooth trays, wherein the protrusions keep the upper teeth of the user at a desired distance from the lower teeth of the user, and wherein adjusting the length of the connectors, and/or the elastic mechanisms, pushes or pulls the user's lower jaw backward or forward with respect to the user's upper jaw, as desired.

2. The oral appliance of claim 1, wherein each partial tooth tray is made of plastic, acrylic, or other synthetic material.

3. The oral appliance of claim 1, wherein each partial tooth tray includes three planes that form an open channel and, at least partially, cover three sides of a tooth.

4. The oral appliance of claim 3, wherein at least one plane of each partial tooth tray is replaced by at least one wire or pre-fabricated or custom made bar or connector in that plane.

5. The oral appliance of claim 1, wherein one adjustable pad that stays between the user's upper lip and the user's upper frontal gum is attached by wire to one upper partial tray to assist with nasal breathing.

6. The oral appliance of claim 1, wherein the first and the second partial lower tooth trays are attached to each other by a wire or by a pre-fabricated or custom made bar or connector and/or the first and the second partial upper tooth trays are attached to each other by a wire or by a pre-fabricated or custom made bar or connector.

7. An oral appliance for facilitating breathing of a user, the oral appliance comprising:
an upper partial tooth tray to cover upper molars and/or premolars of the user, wherein the upper partial tooth tray does not extend to the upper frontal teeth of the user;
a lower partial tooth tray to cover lower molars and/or premolars of the user, wherein the lower partial tooth tray does not extend to the lower frontal teeth of the user;
a first adjustable-length connector, and/or an elastic mechanism, attached on the right side of the user's mouth between a rear and/or a front side of the upper partial tooth tray to a rear and/or a front side of the lower partial tooth tray;
a second adjustable-length connector, and/or an elastic mechanism, attached on the left side of the user's mouth between a rear and/or a front side of the upper partial tooth tray to a rear and/or a front side of the lower partial tooth tray;
an adjustable or a non-adjustable height protrusion from the upper partial tooth tray or the lower partial tooth tray such that the protrusion keeps the upper teeth of the user at a desired distance from the lower teeth of the user, and wherein adjusting the length of the adjustable-length connectors, and/or an elastic mechanisms, pushes or pulls the user's lower jaw backward or forward with respect to the user's upper jaw, as desired or as needed.

8. The oral appliance of claim 7, wherein each partial tooth tray is made of plastic or acrylic or any other synthetic material.

9. The oral appliance of claim 7, wherein each partial tooth tray form an open channel and, at least partially, covers three sides of molars and/or premolars of the user.

10. The oral appliance of claim 7, wherein each partial tooth tray form an open channel and, at least partially, covers three sides of molars and/or premolars of the user, and wherein at least one side of the open channel is made of retentive wires.

11. The oral appliance of claim 7, wherein an adjustable pad that stays between the user's upper lip and the user's upper frontal gum is attached by wire to the upper partial tray to assist with nasal breathing.

12. The oral appliance of claim 7, wherein a right and a left side of the upper and/or the lower partial tooth trays are attached to each other by a narrow metal connector, pre-fabricated or custom made bar or connector.

13. A method of managing breathing of a user, the method comprising:
placing a first and a second partial upper tooth trays over upper molars and/or premolars of the user, wherein the upper partial tooth trays do not extend to the upper frontal teeth of the user;
placing a first and a second partial lower tooth trays over lower molars and/or premolars of the user, such that the first partial lower tooth tray is substantially located under the first partial upper tooth tray and the second partial lower tooth tray is located under the second partial upper tooth tray, wherein the lower partial tooth trays do not extend to the lower frontal teeth of the user;
adjusting a length of a first adjustable-length connector attached between a rear side of the first partial upper tooth tray to a front side of the first partial lower tooth tray or between a front side of the first partial upper tooth tray to a rear side of the first partial lower tooth tray;
adjusting a second adjustable-length connector attached between a rear side of the second partial upper tooth tray to a front side of the second partial lower tooth tray or between a front side of the second partial upper tooth tray to a rear side of the second partial lower tooth tray, wherein adjusting the length of the connectors pushes or pulls the user's lower jaw backward or forward with respect to the user's upper jaw, as desired; and
keeping the upper teeth of the user at a desired distance from the lower teeth of the user by attaching an adjustable or a non-adjustable height protrusion to the first and/or the second partial upper tooth trays or to the partial lower tooth trays.

14. The method of claim 13, wherein each partial tooth tray is made of plastic or acrylic or other appropriate synthetic material.

15. The method of claim 13, wherein each partial tooth tray includes three planes that form an open channel and, at least partially, cover three sides of a tooth.

16. The method of claim 15, wherein at least one plane of each partial tooth tray is replaced by at least one retentive wire in that plane.

17. The method of claim 13, wherein one pad that stays between the user's upper lip and the user's upper frontal gum is attached by wire to each upper partial tray to assist with nasal breathing.

18. The method of claim 13, wherein the first and the second partial lower tooth trays are attached to each other by a wire, a pre-fabricated or custom made bar or connector and/or the first and the second partial upper tooth trays are attached to each other by a wire, a pre-fabricated or custom made bar or connector.

19. The method of claim 13, wherein the adjustable-length connectors are attached to the partial tooth trays by ball and socket joints.

20. The method of claim 13, wherein the adjustable-length connectors are attached to the partial tooth trays by a movable joint or wherein the adjustable-length connectors are elastic or spring and are attached to the partial tooth trays by connector hooks.
